(19) United States
(12) Patent Application Publication
(10) Pub. No.: US 2009/0143649 A1
(43) Pub. Date: Jun. 4, 2009

(54) Speculum for the electropharmacological treatment of vaginal diseases

(75) Inventor: Cino Rossi, Rome (IT)

Correspondence Address: COLLARD & ROE, P.C., 1077 NORTHERN BOULEVARD, ROSLYN, NY, 11576, US

(73) Assignee: Physion SRL

(21) Appl. No.: 11/998,522

(22) Filed: Nov. 30, 2007

(51) Int. Cl. A61B 1/32; A61N 1/30

(52) U.S. Cl. 600/221; 604/21

(57) ABSTRACT

A speculum for the electropharmacological treatment of vaginal diseases has a pair of elongated blades interconnected such that an opening adapted for observation and passage of instruments is defined between the blades. There is at least one electrode connected to one of the blades on an inner face of the blade. The electrode has a proximal insulated portion and a distal uninsulated portion. To treat vaginal diseases, the speculum is placed in a vagina so that the electrode is located proximate to the vaginal disease, and a drug solution is injected into the vagina. An electric current is then applied to the electrode, which causes the migration of the drug solution toward the vaginal disease.

### BACKGROUND OF THE INVENTION

1. Field of the Invention

The present invention relates to a speculum for the electropharmacological treatment of vaginal diseases and to the related Electromotive Drug Administration (EMDA) for treatment of uterine and vaginal diseases.

2. Description of the Prior Art

The present invention originates from the statistical observation that the number of new cases of diseases of the feminal genital tract, such as severe infections, vulvo-vaginitis, premalignant and malignant changes of the vagina, vulva, cervix and neoplasia has considerably increased in the last few years, in the industrial countries. Furthermore, a number of minor vaginal infections which commonly affect a high number of women during their life require intensive treatments and, sometimes, hospitalization.

Accordingly, a number of instruments in the field of preventive medicine, meant for routine examinations and periodical check-ups, such as for obtaining Papanicolau of flux samples and other vaginal examinations, have been recently developed.

In the area of gynaecological observations and minor operations, vaginal specula, devices made of two tong-like spreading members which are pivotally interconnected and which include an inspection window in handle portions integral with the spreading members, are commonly used.

The specula according to the prior art are made of metal, usually stainless steel, such that they could be reused or, in the alternative, made of a pre-sterilized disposable material such as a thermoplastic material.

The vaginal speculum is typically employed to dilate the vaginal cavity so that an examination of the cervix and vaginal tissues may be readily performed.

In the area of severe gynaecological treatments, there have been proposed a variety of locally tissue-destructive techniques for treating pre-or malignant affections, such as hysterectomies, surgical treatment of cervical cancers, local excision cryotherapy, electrocautery. In some cases, more extensive surgical procedures are used, involving indiscriminate destruction or surgical removal of the entire organ.

In these severe cases, local administration and passive diffusion of anticancer drugs have proven to be ineffective in the vaginal cavity due to an ineffective rate of diffusion of the drugs into the targeted tissues. Therefore, some additional force is required to achieve an effective pharmacological local treatment of the more severe cases.

Furthermore, assuming that this force is made available, a specific device ad equipment are required in order to perform, at a same time, both a diagnostic and a therapeutical treatment of feminal anatomic target sites: vagina, vulva and cervix.

It is also known that the term EMDA refers to the electromotive drug administration, an electropharmacological treatment in which three important electrokinetic phenomena are involved, namely: iontophoresis, electrophoresis and electroporation. lontophoresis is defined as the active transport of ionised molecules into tissues by application of an electric current through a solution containing the ions to be delivered.

Electroctrophoresis refers to the transport of solutes associated with bulk movement of water.

Electroporation describes a phenomenon according to which the application of an electric field causes an increase in the permeability of biological membranes, achieving an increased transport of drugs down concentration gradient because the value of the diffusion coefficient has been increased.

Electromotive drug delivery and related iontoelectrophoretic phenomena are disclosed for example in U.S. Pat. No. 5,301,688.

Although electromotive drug administration to the body has been in existence for more than 80 years, a specific application in the medical field in conjunction with a gynaecological diagnostic method is, at present, unknown.

### SUMMARY OF THE INVENTION

It is one of the principal aims of the present invention to provide a device having use in gynaecological medicine, which can be employed to carry out both routine examinations or periodical check-ups and treatments on the diagnostic disease.

It is another aim of the present invention to provide a device having design and structural features that make it adapted for use both in the field of preventive medicine for gynaecological observations and in the field of electro-treatments of vaginal diseases.

It is yet another aim of the present invention to provide a device to be used in the outpatient treatments of some vaginal, cervical, uterine, vulvar diseases, without resorting to the radical approach of conventional treatments and yet which will be effective.

It is a further aim of the present invention to provide a method both for alleviating pain at the pelvic region of the parturient and for achieving an effective dilatation of the cervix uteri to facilitate travail.

Other objects of the present invention will become apparent from a consideration of the description, accompanying drawings and appended claims.

With the foregoing and other objects in view, there is provided, in accordance with the present invention, a speculum for the electropharmacological treatment of vaginal diseases, said speculum comprising a pair of elongated blades interconnected such that an opening adapted for observation and passage of instruments is defined between said pair of blades at an end region of said blades, at least one electrode being connected to one of said elongated blades. Said electrode is preferably provided on an inner face of one of said elongated blades and has a proximal insulated portion and a distal uninsulated portion.

In accordance with an embodiment of the invention, each of said pair of elongated blades is provided with an electrode having a proximal insulated portion an a distal uninsulated portion, advantageously having a spiral or flat configuration.

In accordance with a further embodiment, the speculum of the invention includes handles, advantageously provided with means for regulating and adjusting the separation of said blades and for their retention in a selected opened position.

According to a further embodiment of the invention, the speculum is provided, between the operating section and the vulva, with hydrophilic fabric, like cotton wool, so as to allow a constant soaking of the pharmaceutical solution injected into the vagina. Said fabric can also extend along the length of the two blades, thus easing the flowing of the pharmaceutical solution towards the vulva itself and providing a solution to the problems caused by the force of gravity, which makes it difficult to obtain a uniform distribution of the pharmaceutical solution between the upper and the lower portions of the part to be treated, the upper part being usually left on a dryer state.

According to a further embodiment of the invention, the surfaces of the two blades are provided with many small holes, preferably having a diameter ranging from 1 to 2 millimetres, which allow the passing of the pharmaceutical liquid.

According to yet another embodiment of the invention, the inner parts of the two blades are removed and each blade is reduced to a U-shaped semi-tubular element following its original contours. The semi-tubular element is substantially hollow but in the end part, so that the electrode may be easily inserted into any of them and realised after use.

According to another aspect of the same embodiment, the U section of the semi-tubular element may be cut out in the middle section, and the edges rounded so as to improve the flexibility of the protruding parts that are inserted into the vagina.

In accordance with another aspect, the present invention provides for a method of treating vaginal diseases by localized electromotive drug administration, comprising the steps of:
placing, along a vagina, a speculum comprising a pair of elongated blades interconnected such that an opening adapted for observation and passage of instruments is defined between said pair of blades at end region of said blades, and having at least one electrode connected to one of said elongated blades;
adjusting said pair of elongated blades in an opened position to check throughout said opening the position of a vaginal disease;
positioning said electrode proximate to said vaginal disease;
infusing a solution of a drug advantageously selected among drugs to treat said vaginal disease, by means of injection means;
applying an external counter-electrode at a convenient body region;
electrically connecting said electrode and counter-electrode to an external circuit that includes an electric current source, and
applying to said electrodes an electric current suitable to cause the migration of said solution of a drug toward said vaginal disease.

The term “drug selected to treat a vaginal disease” relates to any type of drug useful for treating a disease at the vaginal area by electromotive administration.

Selection of the drug will depend on the type of disease which affects the patient in need of treatment, preferred drugs being selected from the group consisting of anticancer, analgesic, anaesthetic, local, antibiotic, antifungine, vasodilator, vasoconstrictor, anti-inflammatory, utero-tonics, hormones and haemostatic drugs and mixtures thereof.

In accordance with an embodiment of the present method of treatment, the electrode connected to said elongated blades is an anode. During use said anode is provided along a vaginal cavity to promote an electromotive drug administration of a positively charged drug.

In accordance with this embodiment, a cathodic counter-electrode connected to an external power source is provided, to close the electric circuit.

In accordance with a further aspect of the present invention a method for dilating a uterine canal by a localized electromotive drug administration is provided, said method comprising the steps of:
placing, along a vagina, a speculum comprising a pair of elongated blades interconnected such that an opening adapted for observation and passage of instruments is defined between said blades, at least one electrode being connected to one of said elongated blades,
inserting throughout the opening of said speculum and within said vagina a catheter having a flexible tubular body including a plurality of openings preferably at its distal portion, said catheter being internally provided with a second electrode,
positioning the distal portion of said catheter along the uterine canal,
infusing via said catheter an electrically conductive solution of a drug advantageously selected from the group consisting of anaesthetic local drug, sympathomimetic drug, spasmolithic drug and mixtures thereof,
electrically connecting said electrodes and counter-electrode to an external circuit that includes an electric current source, and
applying to said electrodes an electric current suitable to cause the migration of said solution of a drug at said uterine canal.

In accordance with an embodiment of the method of the invention, each of said elongated blades includes an electrode having a distal uninsulated portion and a proximal insulated portion.

The electrode inserted within the catheter can act as counter electrode or as active electrode.

### BRIEF DESCRIPTION OF THE DRAWINGS

The speculum and the performance of the method of the method of treatment of the present invention, together with additional objects and advantages thereof, will be best understood from the following description of specific embodiments thereof, when read in connection with the accompanying drawings, in which:

FIG. 1 is a lateral view of an embodiment of the speculum according to the present invention, wherein each one of the elongated blades is provided with an electrode,

FIG. 2 is an enlarged-scale view showing the lower blade of the present of the speculum of FIG. 1, which is provided with an electrode having a spiral-shaped conductive portion,

FIG. 3 is an perspective view of the embodiment shown in FIG. 1, illustrating that the two electrodes connected to the elongated blades have a proximal insulated portion directed to a power source (not shown) and an uninsulated portion to provide an electromotive drug administration to a target area of a vagina,

FIG. 4 is a front view of the speculum of FIG. 1, showing the opening of the vaginal speculum, said opening being provided to perform both a vaginal exploration and the method of treatment according to the invention,

FIG. 5 shows the embodiment of FIG. 1 during the performance of a method for the dilatation of a uterine canal by electromotive drug administration.

### DESCRIPTION OF THE PREFERRED EMBODIMENTS

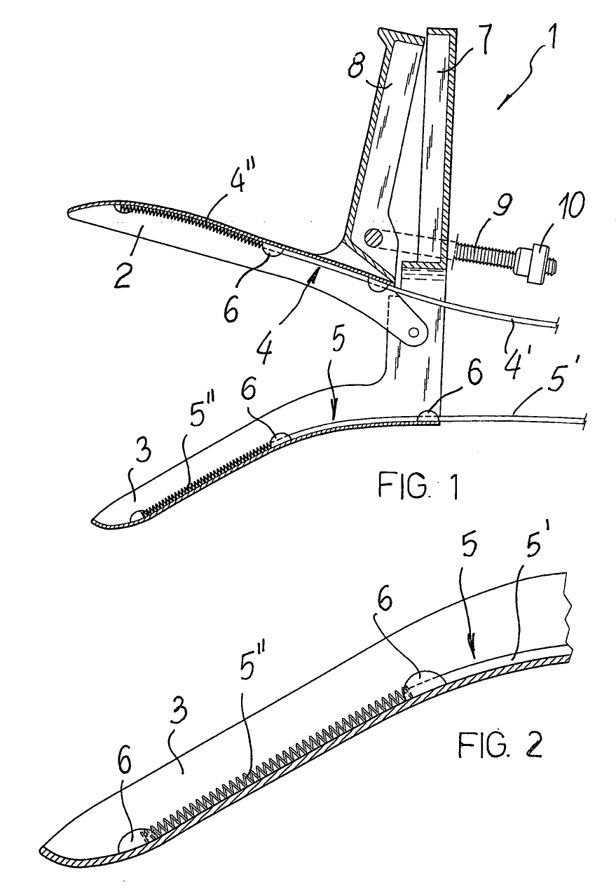

Referring now to the drawings and initially to FIG. 1, a vaginal speculum 1 in accordance with the present invention is shown. The speculum 1 comprises two elongated blades 2, 3 which extend facing each other and which are provided, within their inner surface, with electrode 4, 5.

The electrodes are connected to said blades by connection means or adhesive means. Each electrode 4, 5 is divided into a proximal insulated portion 4′, 5′, directed to a power source (not shown) and an uninsulated portion 4″, 5″ which extends along a portion of said blades 2, 3. In this preferred embodiment, the blades 2, 3 are pivotally interconnected and are also provided with handles 7, 8. Said handles 7, 8 comprise means 9, 10 for adjusting the distance between the blades 2, 3 and for retaining them in a suitable position.

FIG. 1 illustrates the speculum 1 with the blades 2, 3 in an opened position, as required in the vaginal inspection and in the performance of the electropharmacological method of treatment of the invention. The section of each one of the blades 2, 3 is concave-convex, adopting a suitable shape corresponding to the anatomy of the vaginal cavity.

The speculum 1 is further provided with handles 7, 8 having a threaded rod 9 whereon a nut 10 may be operated for adjusting and retaining the blades 2, 3 when they are positioned along a vagina, in a given angle selected by the acting gynaecologist.

Advantageously, the speculum 1 is made of metal such as stainless steel or of plastic material, in this case advantageously of the disposable type.

Referring to FIG. 2, the inside face of the blade 3 is provided with an electrode 5 which, during use, promotes the electromotive administration of an ionized drug solution to the target tissue. This electrode 5 is of the same type as provided at the other blade 4. The uninsulated portion 5″ of the electrode 5 is spiral-shaped, since this configuration allows a very effective diffusion of a flow of electrical current, in an ionised environment.

The electroconductive portion 5″ of the electrode 5 can be made of any conductive material and can act as an active or a passive electrode, depending on the type of material being used.

Suitable conductive materials are selected from the group consisting of stainless steel, aluminium, copper, nickel, titanium, zinc, gold and mixtures thereof.

The conductive portion 5″ is preferably made of tempered stainless steel, since this material entails the desirable qualities of strength, resilience and durability.

Insulation of the electrode 5 from the power source to its uninsulated portion 5″ (spiral filamentous portion) is achieved by using polymer coatings, such as thermoretractive polyurethane or Teflon. The surface of the uninsulated portion 5″ is made of stainless steel or is coated with a suitable conductive material deemed best at the fluid interface.

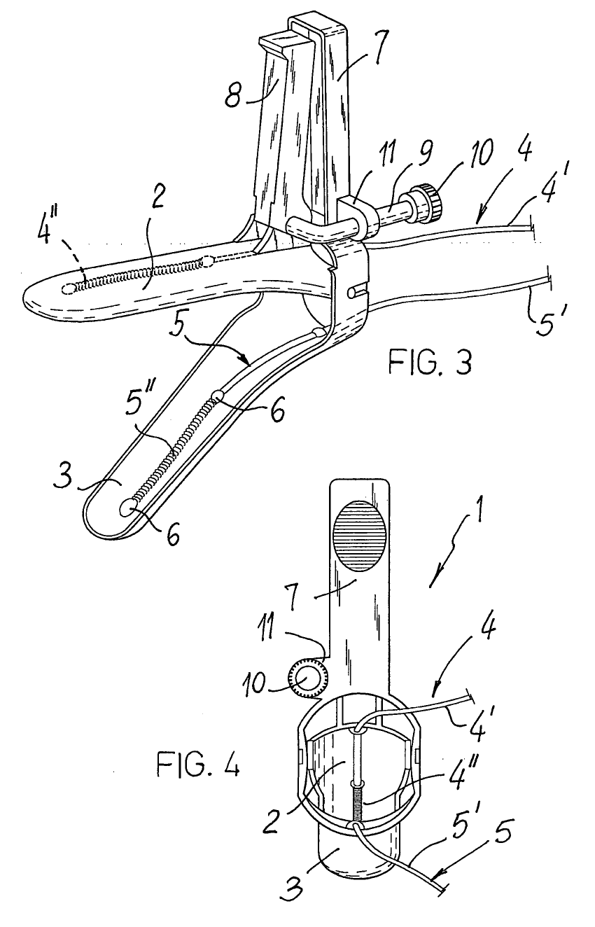

The adjusting means 9, 10 of the speculum 1 may be embodied in various practical forms, such as the one illustrated in details in FIG. 3, consisting of a rod 9 threaded in its median an upper portions and connected to the lateral wall of a second handle 8.

A first handle 7 is provided with an integral annular ring 11 which defines a passage for said rod 9. A nut 10 may be screwed in or out to achieve and maintain the required distance between the blades 2, 3.

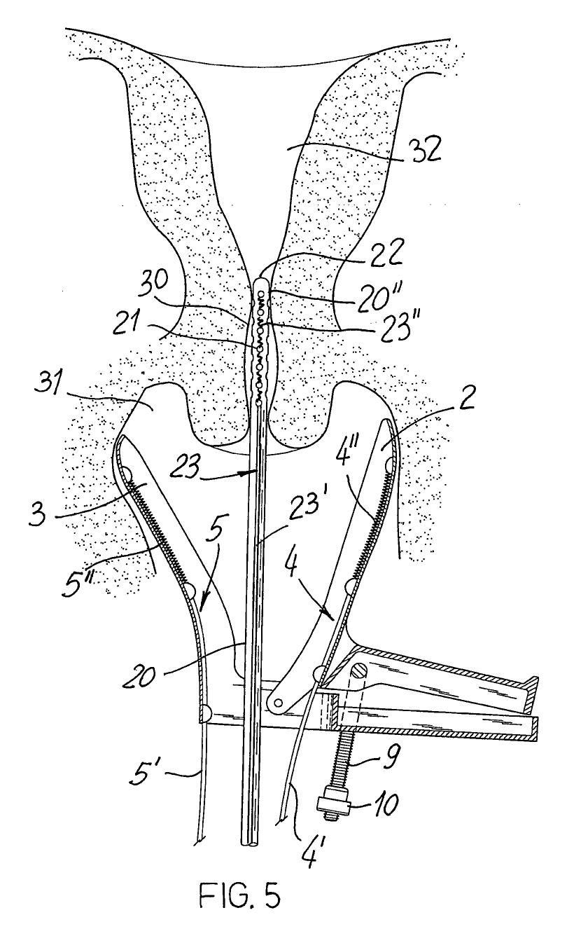

FIG. 4 illustrates an opening 12 of the speculum 1 adapted for observation and diagnosis of a disease affecting a vaginal cavity. Said opening 12 may be also provided with means (not shown) for supporting instruments to be placed inside a vaginal cavity, and for locking in a pre-fixed position.

In accordance with the therapeutical method of the invention, the free ends of said blades 2, 3 are inserted, in a closed position, within a vagina of a patient, whereafter the physician may act on the handles 7, 8 to slowly separate said blades 2,3 from each other until the field of observation through the opening is wide enough.

The blades 2, 3 are then precisely positioned within the vaginal cavity, at a predetermined distance from the diseased tissue.

In particular, the uninsulated portion 4″, 5″ of the electrodes 4, 5 is placed proximate to the affected tissue, whereas the insulated portion 4′, 5′ is connected to an external power source (not shown). Such power source is connected to a secondary dispersive electrode (not shown) placed on some convenient area of the skin of a patient in need of treatment.

A drug solution is then instilled through said opening within said vaginal cavity, via an injection means placed through said opening.

The power source is then switched on, activating an ion flow contained in the drug solution.

The application of an electric current to the drug solution advantageously ensures that the rate of diffusion to the affected vaginal tissue is accelerated. The ion flow can be controlled in a simple way, by varying the time of treatment and the intensity of the electric field, in a range between 5 to 80 mA, preferably between 10 to 40 mA.

The power source useful in the method of the invention is preferably of the current-controlled type, more preferably a battery-powdered source. The current waveform incorporates constant DC, pulsed DC and advantageously a timing device to allow reversal of polarity with a prearranged frequency.

Following application of electric current from the power source, drug ions are repelled into and through the diseased tissues, where they will attach to their specific receptors. The method according to the present invention is suitable both in the diagnostic and in the treatment of a wide range of diseases affecting the vaginal cavity. For example, the method of the invention is useful in treating vaginal, cervical, uterine, vulvar neoplasiae, severe vaginomycosis, infections, pre-malignant epithelial lesions, vaginitis, vulvo-vaginitis, cervical-vaginitis, utero-vaginitis, etcetera.

The drug solution administered by EMDA comprises one or more active ingredients considered more suitable for treating the diagnostic disease. The drugs are selected because of their desirable pharmacological properties. Such drugs are advantageously supplied in the form of a charged or non-charged salt.

By way of example, the method of treatment of the invention is useful for performing local anticancer therapy by the electromotive administration of anticancer agents such as mitomycin C, doxorubicin, mythramycin, mitoxantrone and other ionisable anticancer agents.

The localized administration of these toxic active ingredients at vaginal targeted malignant or pre-malignant tissues is not only highly effective but also considerably reduces the problems of general toxicity and related side effects.

When an anaesthetic drug is administered, such drug is preferably infused as solution in distilled water.

When a anaesthetic agent is not used, physiological electrolyte solutions including Na⁺, K⁺, Ca²⁺, Cl⁻, Lactate⁻, emisuccinate⁻ ions, are locally administered, in concentrations preferably ranging from 150 mM to 350 mM. In the alternative, sodium/potassium citrate salt solutions are useful as electrolyte solution, advantageously provided at concentrations of 1 to 3% by weight.

By way of another example, lidocaine, mepivacaine, bupivacine, ropivacaine, ketocaine at a suitable concentration of 0.3 to 3% by weight are administered in conjunction with epinephrine, to a concentration ranging 1/200,000- 1/50,000.

FIG. 5 illustrates the embodiment of FIG. 1, as it may be used in a practical execution of a method for dilating a uterine canal by the localized electromotive drug administration according to the invention.

The use of the vaginal speculum 1 provides for the preliminary dilatation of a vaginal cavity 31 of a patient in need of dilating an uterus 32. The dilatation of the vaginal cavity allows the gynaecologist to check the status of the cervix uteri.

The following step comprises the insertion of a catheter 20 within a uterine canal 30 in need of treatment.

The catheter 20 useful in the present method may be one of available urethral catheters, e.g. a Foley-type catheter, and is advantageously provided with a plurality of holes 21 and its distal portion 20′ and with a rounded tip 22. Said holes are placed and maintained, during the treatment, within the uterine canal to be dilated. The catheter may be further provided with elastic sealing means, like an inflatable balloon, so as to provide optimum insulation.

A stem-like counter electrode 23 having a distal uninsulated portion 23″ and a proximal insulated portion 23′ passes inside the hollow tubular body of said catheter. The uninsulated portion 23″ of the electrode 23 extends along the distal portion 20″ of said catheter 20, wherein openings 21 are provided, to give a current flow at the uterine canal to be treated.

The catheter may be further provided with elastic sealing means, like an inflatable balloon, so as to provide optimum insulation.

An external power source (not shown) is connected to a secondary dispersive electrode (not shown) placed on some convenient area of the skin of the patient to be treated.

An electrically conductive drug solution is then instilled in the uterine canal 30, via said catheter 20. At the same time, the electric power source is activated, to close the electric circuit.

A current flow having an intensity of 5 to 50 mA is applied to the areas where the conductive solution is present.

Under this specific conditions, a ion flow is directed to the targeted body areas (uterine canal and surrounding tissues), providing for a high drug concentration only proximate to the tissues to be treated.

The infused solution includes as active ingredients a ionisable drug selected from the group consisting of anaesthetic drug, sympathomymetic agents, anti-inflammatory drugs and mixtures thereof.

For example, a local anaesthetic drug providing in solution a positively charged ion is used in concomitance with a positively charged electrode 23. Said electrode 23 acts by repelling the positively charged anaesthetic ions through the tissue in need of treatment and into the specific sodium-potassium channels, along the axons of parasympathetic nerves, where said anaesthetic drugs induce a reversible blockade of conduction along said nerves. The duration of action of the local anaesthetic drug mainly depends on the particular drug being selected.

The electromotive drug administration according to the present invention is of particular value during partum, since it provides for both an analgesic effect at the pelvic region and a profound relaxation of the muscular tissues proximate to the uterine canal.

The local electromotive drug administration is further highly appreciable when performed onto a pregnant, since in this condition the systematic absorption of the drugs and the following delivery to the fetus are avoided.

1. A speculum for the electropharmacological treatment of vaginal diseases, comprising a pair of elongated blades mutually connected such that an opening adapted for observation and passage of instruments is defined between said pair of blades at an end region of said blades, at least one electrode being connected to one of said elongated blades.

2. A speculum according to claim 1, wherein said electrode is provided on an inner face of one of said elongated blades and has a proximal insulated portion and a distal unisulated portion.

3. A speculum according to claim 1, wherein each of said elongated blades is provided with an electrode having a distal uninsulated portion and a proximal insulated portion.

4. A speculum according to claim 3, wherein said uninsulated portion of the electrode has a spiral configuration.

5. A speculum according to claim 1, wherein said electrode comprises an uninsulated portion made of a conductive material selected from the group consisting of stainless, aluminium, copper, nickel, titanium, zinc, nitinol, silver, gold, carbon and mixtures thereof.

6. A speculum according to claim 1, further comprising handles, wherein means for regulating and adjusting the separation of said blades and for their retention in a selected opened position are provided.

7. A speculum according to claim 1, wherein said blades are pivotally interconnected.

8. The speculum according to claim 1 wherein the surfaces of said elongated blades are provided with a plurality of holes.

9. The speculum of claim 8 wherein the diameters of said holes range between 1 and 2 millimetres.

10. The speculum according to claim 1, characterized in that said speculum is provided with hydrophilic fabric.

11. The speculum according to claim 10, wherein said hydrophilic fabric extends along the length of said elongated blades.

12. The speculum according to claim 1 wherein said instrument is a catheter provided with a plurality of holes at one distal portion.

13. The speculum according to claim 12 wherein an elastic sealing means is arranged perimetrically around the stem of the catheter.

14. The speculum of claim 13 wherein said elastic sealing means is an inflatable balloon.

15. A speculum for the electropharmacological treatment of vaginal diseases, comprising a pair of U-shaped protruding elements mutually connected so that an opening adapted for observation and passage of instruments is defined between said pair of protruding elements at an end region of said protruding elements, at least one electrode being connected to one of said protruding elements.

16. The speculum according to claim 15 wherein said electrode is provided on an inner face of one of said protruding elements and has a proximal insulated portion and a distal uninsulated portion.

17. The speculum according to claim 16 wherein each of said protruding elements is provided with an electrode having a distal uninsulated portion and a proximal insulated portion.

18. The speculum according to claim 17, wherein said electrode comprises an uninsulated portion made of a conductive material selected from the group consisting of stainless steel, aluminium, copper, nickel, titanium, zinc, silver, gold, carbon and mixtures thereof.

19. The speculum according to claim 15, further comprising handles, wherein means for regulating and adjusting the separation of said blades and for their retention in a selected opened position are provided.

20. The speculum according to claim 15, wherein said protruding elements are pivotally interconnected.

21. The speculum according to claim 20 wherein an elastic sealing means is arranged perimetrically around the stem of the character.

22. The speculum of claim 21 wherein said elastic sealing means is an inflatable balloon.

23. A speculum for the electropharmacological treatment of vaginal diseases, comprising a pair of protruding elements, each element being composed of two curved rods, mutually connected so that an opening adapted for observation and passage of instruments is defined between said pair of protruding elements at an end region of said protruding elements, at least one electrode being connected to one of said protruding elements.

24. A method of treating vaginal diseases by localized electromotive drug administration, comprising the steps of:
placing, along a vagina, a speculum comprising a pair of elongated blades interconnected such that an opening adapted for observation and passage of instruments is defined between said pair of blades at end region of said blades, and having at least one electrode connected to one of said elongated blades, along a vagina;
adjusting said pair of elongated blades in an opened position to check throughout said opening the location of a vaginal disease;
positioning said electrode proximate to said vaginal disease;
infusing a solution of a drug selected to treat said vaginal disease, by means of injection means;
applying an external counter-electrode at a convenient body region;
electrically connecting said electrode and counter-electrode to an external circuit that includes an electric current source, and
applying to said electrodes an electric current suitable to cause the migration of said solution of a drug toward a vaginal disease.

25. A method according to claim 24, wherein said drug is selected from the group consisting of anticancer, analgesic, anaesthetic, local, antibiotic, antifungine, haemostatic, vasodilator, anti inflammatory, haemostatic drugs and mixtures thereof.

26. A method according to claim 24, wherein said electric current is of 10 to 40 mA.

27. A method according to claim 24, wherein the electrode connected to said elongated blades is an anode and the counter-electrode is a cathode, said anode being provided inside said vaginal cavity to promote an electromotive drug administration of a positively charged drug.

28. A method according to claim 24, wherein said solution of a drug includes a solvent selected from the group consisting of distilled water and a physiological electrolyte solution.

29. A method for dilating a uterine canal by a localized electromotive drug administration, comprising the steps of:
placing, along a vagina, a speculum comprising a pair of elongated blades interconnected such that an opening adapted for observation and passage of instruments is defined between said pair of blades at end region of said blades, at least one electrode being connected to one of said elongated blades;
inserting throughout the opening of said speculum and along said vagina a catheter having a flexible tubular body including a plurality of openings at its distal portion, said catheter being internally provided with a counter-electrode,
positioning the distal portion of said catheter along an uterine canal,
infusing via said catheter an electrically conductive solution of a drug selected from the group consisting of anaesthetic local drug, sympathomimetic drug, spasmolithic drug and mixtures thereof,
electrically connecting said electrodes and counter-electrode to an external circuit that includes an electric current source, and
applying to said electrodes an electric current suitable to cause the migration of said solution of a drug at said uterine canal.

30. A method according to claim 29, wherein said drug consists of a mixture of lidocaine and epinephrine.

31. A method according to claim 29, wherein said electric current is applied for a time ranging from 3 to 40 minutes and said electric current is of 10 to 40 mA.

32. A method according to claim 29, wherein each of said pair of elongated blades is provided with an anodic electrode having a distal uninsulated portion and a proximal insulated portion and said drug is a positively charged ion in solution.
